(19) United States
(12) Patent Application Publication
(10) Pub. No.: US 2007/0134259 A1
(43) Pub. Date: Jun. 14, 2007

(54) Methods and compositions for pharmacologially controlled targeted immunotherapy

(76) Inventors: David Bundle, Edmonton (CA); Pavel Kitov, Edmonton (CA); Gordon Grant, Edmonton (CA); Tomek Lipinski, Edmonton (CA); Sebastian Dziadek, Edmonton (CA)

Correspondence Address: NIXON & VANDERHYE, PC, 901 NORTH GLEBE ROAD, 11TH FLOOR, ARLINGTON, VA, 22203, US

(21) Appl. No.: 11/602,493

(22) Filed: Nov. 21, 2006

Related U.S. Application Data
(60) Provisional application No. 60/738,043, filed on Nov. 21, 2005.

(51) Int. Cl. A61K 39/00; A61K 38/12; A61K 31/739; A61K 31/715; A61K 31/655

(52) U.S. Cl. 424/184.1; 514/54; 514/9; 514/156

(57) ABSTRACT

The present invention relates generally to methods and compositions for targeted immunotherapy. More specifically, the present invention relates to immuno-targeted therapies, using heteromultivalent compounds to mediate the binding of an endogenous effector molecule such as an antibody to target molecules including malignant cells and tissues, bacteria and viruses as well as their toxic agents.

### PRIORITY

The present invention claims the benefit of priority from U.S. patent application 60/738,043 filed Nov. 21, 2006.

### FIELD OF THE INVENTION

The present invention relates generally to methods and compositions for targeted immunotherapy. More specifically, the present invention relates to immuno-targeted therapies using heteromultivalent compounds to mediate the binding of an antibody to target molecules including receptors on malignant cells and tissues, bacteria and viruses as well as their toxic agents.

### BACKGROUND OF THE INVENTION

The traditional direct approach to immunological therapies is the use of antibodies specific to various target cells, particularly cancer cells. Recent advances in tumor specific antigens has led to FDA approval of antibodies (Stem, M. and Herrmann, R. Overview of monoclonal antibodies in cancer therapy. Crit. Rev. Oncol. Hematol. 2005. 54; 11-29).

All of the antibodies approved by the FDA for immunological therapies are IgG based. The success of IgG based therapies is based on the fact that IgG has a high binding constant, making it difficult for the IgG antibody to dissociate from the target cell once bound, and that IgG isotypes are a strong initiator of antibody-dependant complement cytotoxicity, a normal biological immunity event.

It is well known that criteria important for immuno-therapies include: a densely over-expressed cell surface target that is readily distinguishable from healthy somatic cells, a surface target that will not enter the plasma or internalize after binding with an antibody; and the initiation of complement-dependent or cell-mediated cytotoxicity.

A direct immunological strategy for treating cancer can be problematic since not all cancer cells have been demonstrated to have surface antigens distinct from normal tissues (Cavallo, Curico et al. 2005 Immunotherapy and Immunoprevention of Cancer: Where do we stand? 2005. Expert Opinion on Biological Therapy 5(5), 717-726). Other reasons for such difficulty are the inability of high molecular weight molecules to penetrate into the tumor, production of tight intracellular adhesion molecules, the secretion of proteoglycan molecules that non-specifically bind antibodies, and the absence of effector cells inside the tumor.

Accordingly, there continues to be a need for immunotherapies that overcome past problems.

### SUMMARY OF THE INVENTION

It is an object of the present invention to obviate or mitigate at least one disadvantage of current immuno-targeted therapies.

In a first aspect, the present invention provides a novel immuno-targeted strategy for treating various diseases. More specifically, the invention provides a method of targeted immunotherapy comprising administering an effective amount of a compound B having a receptor binding factor (RBF), a synthetic hapten ligand and a linker molecule connecting the RBF and synthetic hapten ligand wherein administration of compound B initiates immune recognition of compound B by pre-existing heterovalent antibodies and wherein the RBF binds a surface receptor of a target and the heterovalent antibodies bind the synthetic hapten ligand. In one embodiment, the target are target cells and compound B promotes antibody-mediated cytotoxicity of transformed target cells. In another embodiment, compound B is administered at a threshold level determined to allow complex formation and activation of antibody-mediated cytotoxicity. In another embodiment, compound B is administered at a dose to promote a multipoint interaction between said antibodies and the target cells.

In a second aspect, the pre-existing heterovalent antibodies are raised in a patient prior to commencing a treatment by administering a compound A, compound A having a carrier, a synthetic hapten ligand and a linker molecule connecting the carrier and synthetic hapten ligand.

In a more specific embodiment, the RBF is Arginine-Glycine-Aspartic Acid (RGD) or a functional derivative or synthetic mimetic thereof. The RGD may be a cyclo-peptide.

In another embodiment, the synthetic hapten ligand is a sulphonamide such as sulfathiazole (STZ). The synthetic hapten ligand may also be any one of nitrophenol, α-(1-3)galactosyl-lactose or ABO blood group antigens.

In other embodiments, the linker molecule may be a heteroatom substituted or un-substituted C2-C20 aliphatic chain, a substituted or un-substituted aromatic, or a polymer.

In yet further embodiments, the carrier may be a non-protein carrier selected to promote an IgM antibody response or a carbohydrate selected to promote an IgM response. The carbohydrate may be dextran or beta-glucan.

In one embodiment, the pre-existing heterovalent antibodies are human anti-blood group A, B or O antibodies or anti-ax-gal antibodies including xenotransplantation or Galili antigen.

In a further aspect of the invention, compound A is administered at a level to maintain a minimum antibody concentration during treatment.

In one model of the invention, the RBF binds Integrin αvβ3 cell surface receptor wherein the RBF binds a sialoglycoprotein associated with a B cell lymphoma. In a more specific embodiment, RBF is a 2,6-linked sialic acid-containing oligosaccharide.

In other models, the RBF is a trisaccharide or a neuraminic acid derivative wherein the RBF binds hemagglutinin-neuraminidase (HN) or wherein the RBF binds viral lectins.

In another aspect, the invention provides a compound for use in immunotherapy comprising a receptor binding factor (RBF), a synthetic hapten ligand and a linker molecule connecting the RBF and synthetic hapten ligand wherein administration of the compound to a system having pre-existing heterovalent antibodies initiates immune recognition of the compound by the pre-existing heterovalent antibodies and wherein the RBF binds a surface receptor of a target and the heterovalent antibodies bind the synthetic hapten ligand. In a more specific aspect, the target is a target cell and the compound promotes complement-mediated cytotoxicity of transformed cells.

In more specific embodiments of the compound, the RBF is Arginine-Glycine-Aspartic Acid (RGD) or a functional derivative thereof. The RGD may be a cyclopeptide. The synthetic hapten ligand may be a sulfonamide such as a sulfathiazole (STZ) or a polyacrylamide.

In other embodiments, the linker molecule is a heteroatom substituted or un-substituted C2-C20 aliphatic chain, a substituted or un-substituted aromatic or a polymer.

In yet another aspect, the invention provides a compound for raising heterovalent antibodies comprising a carrier, a synthetic hapten ligand and a linker molecule connecting the carrier and synthetic hapten ligand wherein the carrier is a non-protein carrier that promotes raising an IgM antibody response. In other embodiments, the carrier is a carbohydrate capable of raising an IgM response and/or the carbohydrate is dextran or beta-glucan. In another embodiment, the carrier is a protein carrier that promotes raising an IgG response.

In another aspect of the invention, the invention provides an assay method to determine an optimum concentration range of compound B as defined above, the optimum concentration range of the compound defining a therapeutic window for the use of the compound in immunotherapy, comprising the steps of: a) concurrently incubating i) the compound comprising a receptor binding factor (RBF), a synthetic hapten ligand and a linker molecule connecting the RBF and synthetic hapten ligand together with ii) heterovalent antibodies and iii) an anchored target; b) measuring the concentration of a formed ternary non-covalent complex, the formed ternary complex including the compound, antibody and target; and, c) repeating step b) at varying compound concentration levels to determine an optimum concentration range in which the formed ternary complex is formed. The assay method may be performed wherein the anchored target is a target cell or a purified receptor on the surface of the target cell which is able to bind the RBF of the compound.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 is a schematic diagram of the generalized methodology of the invention;

FIG. 2A is a generalized schematic diagram of Compound A in accordance with the invention;

FIG. 2B is a generalized schematic diagram of Compound B in accordance with the invention;

FIG. 3 is a representative example of a Compound A, namely a sulfathiazole coupled to dextran, in accordance with the invention;

FIG. 4 are representative examples of RGD-IBAITs in accordance with the invention;

FIG. 5 are results showing that the formation of a ternary complex between integrin and anti-STZ rabbit serum is mediated by RGD-STZ IBAIT;

FIG. 5A: Anti-STZ sera binding to integrin coated plate when incubated concurrently with RGD-STZ, IBAIT;

FIG. 5B: Anti-STZ sera binding to integrin coated plate when incubated after incubation of the plate with RGD-STZ, IBAIT;

FIG. 5C: CELISA Anti-STZ sera binding to HTB-14 cell line coated plate when incubated concurrently with IBAIT (triangle series). Corresponding ELISA using integrin coated plate is shown for comparison (square series).

FIG. 6 are representative examples of CD22-IBAITs in accordance with the invention;

FIG. 7 are representative examples of a HN-IBAITs in accordance with the invention; and,

FIG. 8 is a representative NMR of a Compound A including dextran and STZ.

### DETAILED DESCRIPTION

In accordance with the invention, novel immunotherapies and compounds for affecting such immunotherapies are described. More specifically, the invention provides methodologies and compounds that effectively recognize the existence of target cells or compounds and that subsequently enable the destruction or removal of such target cells or compounds through immune response processes. Target compounds may include toxic compounds or cell surface receptors in target cells including bacteria, viruses and cancer cells or their toxic agents.

General Method and Compound Description

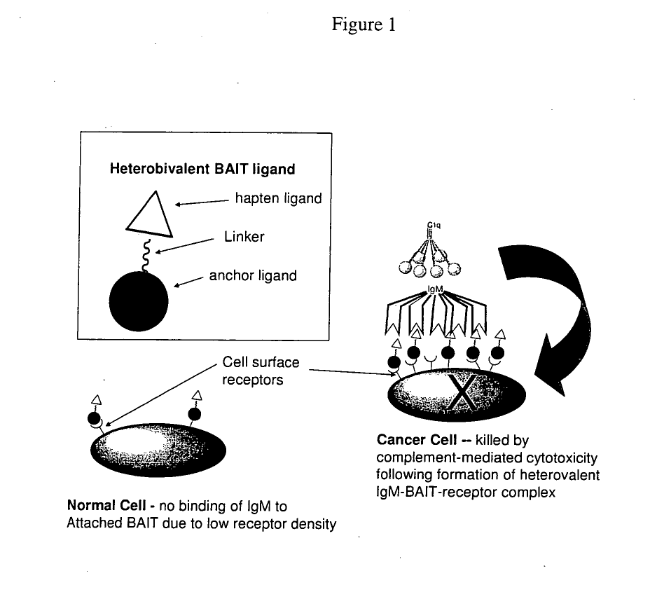

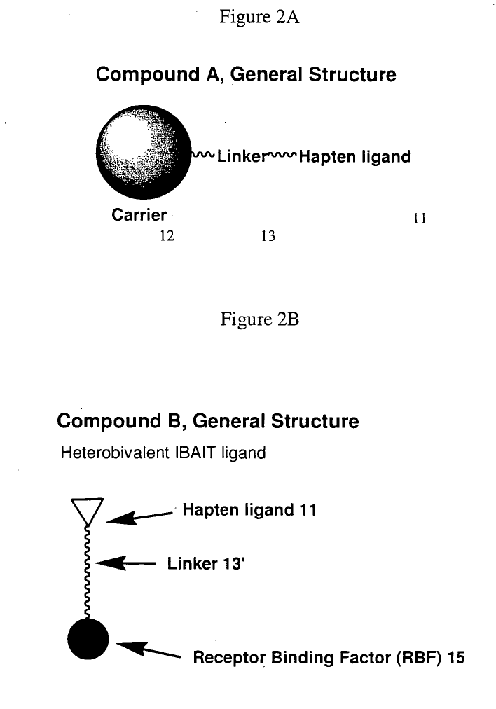

With reference to FIGS. 1-2, the generalized method of the invention is described. Generally, the invention provides a two-step process to effectively target compounds of interest for their removal from a patient.

In the first step, a compound (Compound A) (FIG. 2A) having an immunogenic synthetic ligand component 11, a non-protein carrier 12 and operational linker molecule 13 is administered to a patient to initiate a desired immune response to the synthetic ligand 11 and carrier 12. As a result of this vaccination process, the patient is sensitized to the synthetic ligand 11 and will respond to the future administration of the synthetic ligand 11. The non-protein carrier of Compound A is selected to affect the desired immune response, preferably an IgM response due to higher multivalency. However, if IgG antibody of sufficiently high affinity can be obtained, IgG may substitute for IgM. In the case an IgG response is desired, a protein or peptide may be chosen as a carrier 12.

In an alternate embodiment, the first step may utilize a Compound A ligand that targets pre-existing antibodies normally found in the sera of healthy individuals, such as anti-human blood group A or B antibodies. In such cases the recognition element to be included in the heterobivalent ligand would be the corresponding terminal trisaccharide epitope that binds to the A or B antibodies. Others may include anti-α-gal antibodies (xenotransplantation or Galili antigen).

In the second step, a second compound (Compound B or IBAIT) (FIG. 2B) having a synthetic ligand component 11, a linker component 13′ and a receptor binding factor (RBF) 15 is introduced to the patient. The RBF is a binding factor specific to the target molecule. In the immuno-sensitized patient or patient with the appropriate pre-existing antibody, introduction of Compound B will initiate the immune recognition of the ligand component 11. As a result, Compound B will bind to both the antibodies raised to ligand component 11 as well as the cell surface molecule through the RBF.

As shown in FIG. 1, in the example of cancer cells overexpressing normal surface molecules, the density of such cell surface molecules is higher in the target cells, and thus there is a greater binding of Compound B and antibodies to the cancer cells resulting in enhanced destruction of such cells through the immune processes.

Compound A and IBAIT Selection and Synthesis

From the generalized methodology of indirect immunotherapy, the invention thereby enables the synthesis of specific Compounds A and IBAITs tailored for individual ailments. Specifically, this indirect approach preferably relies upon antibody-mediated complement fixation, recruitment of NK (Natural Killer) cells, monocytes or macrophages to destroy the target cells. IgM antibodies are likely the best candidates as effector molecules in cancer therapy as IgM antibodies are consistently associated with natural immuno-surveillance and subsequent complement-mediated cytotoxicity of transformed cells (Bradlein et al. natural IgM Antibodies and Immuno-Surveillance Mechanisms Against Epithelial Cancer Cells in Humans. Cancer Res. 2003. 63; 7995-8005), but this does not preclude other immunoglobins, like IgG, being successful effector molecules in the IBAIT cancer strategy.

Using a non-cellular effector system addresses some of the problems associated with immune cell penetration of current antibody cancer therapy models. Theoretically, the lower binding constants associated with the IgM isotype can be compensated by multimeric binding of cell surface antigen clusters, which may offer adequate avidity to afford complement associated cytotoxicity.

The multivalent attachment of IgM, as opposed to the bivalent attachment of IgG, represents a first level of discrimination for activation of complement to differentiate normal from malignant cell types. A second level of discrimination is manifested by the requirement of IBAIT, to bring together the antibody and the target cells. IBAIT is ideally administered at a threshold level allowing complex formation and activation of complement cytotoxicity. The system is therefore switched on and off based on the maintenance or withdrawal of optimal concentrations of IBAIT, which clears from the body relatively quickly through the kidneys.

Compound A

FIG. 2A shows the general formula of Compound A.

The synthetic ligand 11 is preferably a low molecular weight, non-toxic, easy to synthesize and conjugate, and immunogenic when conjugated.

In specific embodiments, low molecule weight (MW ˜300 kDa) ligands such as sulfonamides are employed. Sulfonamides have been well studied with approximately 25 of the 5000 available having been used in the fields of agriculture and medicine. Sulfonamides are stable, easy to make and conjugate, immunogenic when conjugated and their excretory metabolism and pharmakinetics are well documented. Other compounds such nitrophenol, α-(1-3)galactosyl-lactose, ABO blood group antigens) may also be used as haptens.

The non-protein carrier 12 of Compound A is selected by the type of immune response desired. Sulfonamides, when conjugated to a protein carrier, elicit a highly immunogenic response, producing mainly IgG antibodies. However, in various embodiments, it is preferred that an IgM response be elicited, to take full advantage of its multimeric binding.

It has been determined that carbohydrate carriers generally do not produce a highly immunogenic response but produce the slower IgM response. Polyacrylamide or other regular polymers, that are not digested by human proteases and that are non-toxic, are effective carrier candidates for the synthetic ligand 11.

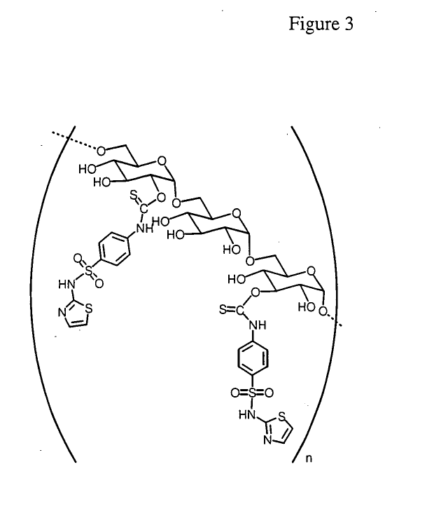

In a specific embodiment, as shown in FIG. 3, an effective Compound A includes a sulfathiazole (STZ) 16, a synthetic ligand, and a Dextran 17, a non-protein carrier. In another embodiment, beta-glucan may also be used as a non-protein carrier.

Naturally occurring haptens, such as alpha-Gal and/or the ABO blood groups, may be used to generate the generic immune response. In further embodiments of the invention, these naturally occurring haptens and the natural humoral response of these haptens obviates the necessity of vaccination with Compound A.

Compound B (IBAIT)

The IBAIT is preferably a relatively small heteromultivalent molecule that can be easily administered as a drug by intravenous or by injection. Compound B is preferably capable of penetrating relevant tissues throughout the human body and be easily removed by the kidneys. The nature of IBAIT's multivalency arises from the assignment of one end of the molecule to determine IgM specificity and the assignment of the other end to target specific over expressed cell surface receptors on various target cells.

RBF

The RBF 15 when coupled to the synthetic ligand 11, must maintain functionality to bind target molecules.

Linker Molecule

The linker molecule in both Compounds A and B is selected to provide sufficient spatial flexibility to both ends of Compounds A and B in order to enable desired binding. The operational linker molecule 13 used in Compound A, may or may not be the same operational linker molecule 13′ used in Compound B. The linker molecule may be an aliphatic or aromatic molecule containing 2-20 atoms of carbon, some of which can be substituted by a heteroatom. An aromatic moiety can be incorporated into an aliphatic chain. The linker can also be polymeric.

### EXAMPLES

### Example 1

### Integrin Model

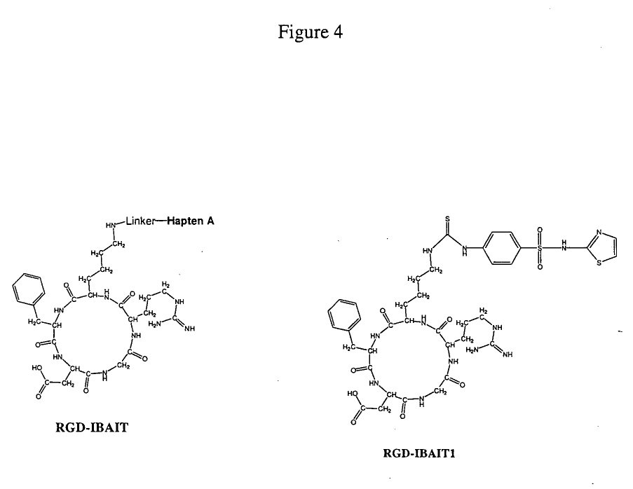

Integrin αvβ3 cell surface receptor is known to be over expressed on cancer cells and/or angiogenesis of vascular tissue associated with tumors. The amino acid sequence of Arginine-Glycine-Aspartic Acid (RGD) (as well as RGD mimetics and functional derivatives) has been shown to have high affinity for integrin αvβ3. As shown in FIG. 4, an IBAIT for treating cancers and/or solid tumors includes a cyclopeptide containing RGD (RGD-IBAIT), as the RBF, and more specifically coupled to STZ (and RGD-IBAIT1). Other amino acid sequences, having a high affinity for integrin αvβ3 cell surface receptors, may also be employed as the RBF to couple the target cells with IgM.

Synthesis of the Cyclic Pentapeptide RGDfK (RGD Peptide)

The cyclic pentapeptide cRGDfK was constructed by the automated assembly of the corresponding protected linear peptide on the solid-phase according to the Fmoc-protocol^{[1]} followed by the cyclization in solution (Scheme 1). For this purpose, the starting amino acid Fmoc-Gly-OH was incorporated onto o-chlorotrityl chloride resin (1) employing DIPEA in dichloromethane. After washing the resin, the protected pentapeptide was assembled through sequential couplings of the corresponding amino acids in a peptide synthesizer. Subsequently, the linear peptide 2 was cleaved from the resin without affecting any of the side-chain protecting groups under mildly acidic conditions using a mixture of acetic acid, 2,2,2-trifluoroethanol and dichloromethane (1:1:3). The head-to-tail cyclization was performed by slowly adding the protected, linear peptide 2 to a solution of 1-propanephosphonic acid cyclic anhydride in ethyl acetate (50%), triethyl amine, and catalytic DMAP in dichloromethane. ^{[2]} High dilution favored the cyclization over the oligomerization yielding 68% of the protected cyclic RGD peptide 3 after column chromatography. The remaining acid-labile side-chain protecting groups were removed with a mixture of trifluoroacetic acid and water followed by purification by RP-HPLC to furnish RGD peptide 4 in 76% yield.

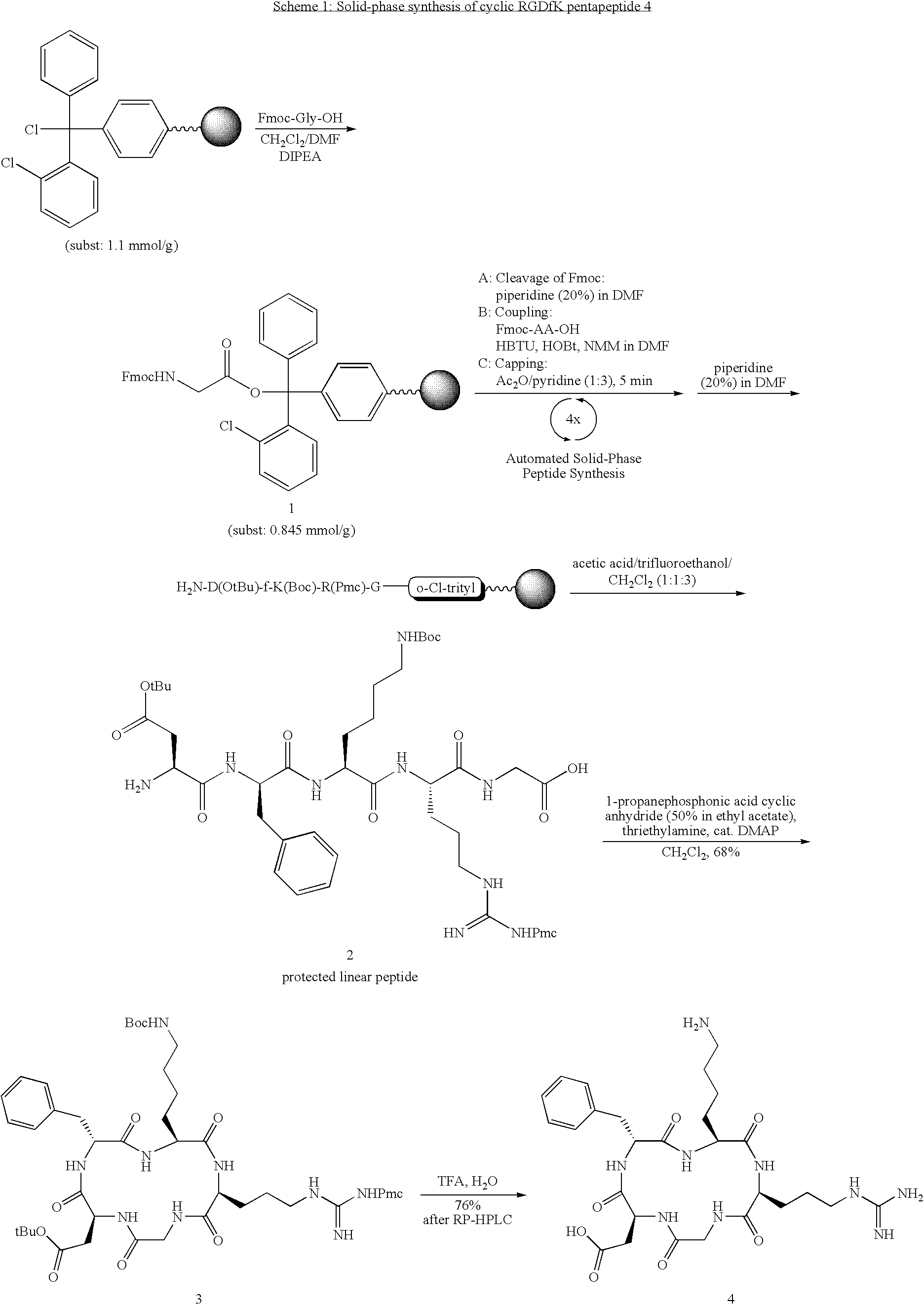

Synthesis of RGD-STZ

The RGD-STZ heterobifunctional ligand system 5 was prepared by conjugating 4-isothiocyanato-N-thiozol-2-yl-benzenesulfonamide, which was readily accessible through the reaction of commercially available sulfathiazole with thiophosgene, to the RGD cyclic peptide 4 via its primary amino functionality (Scheme 2). The reaction was carried out in DMF using N-methylmorpholine as the base. After purification by preparative RP-HPLC, the target compound 5 was obtained in a yield of 67%.

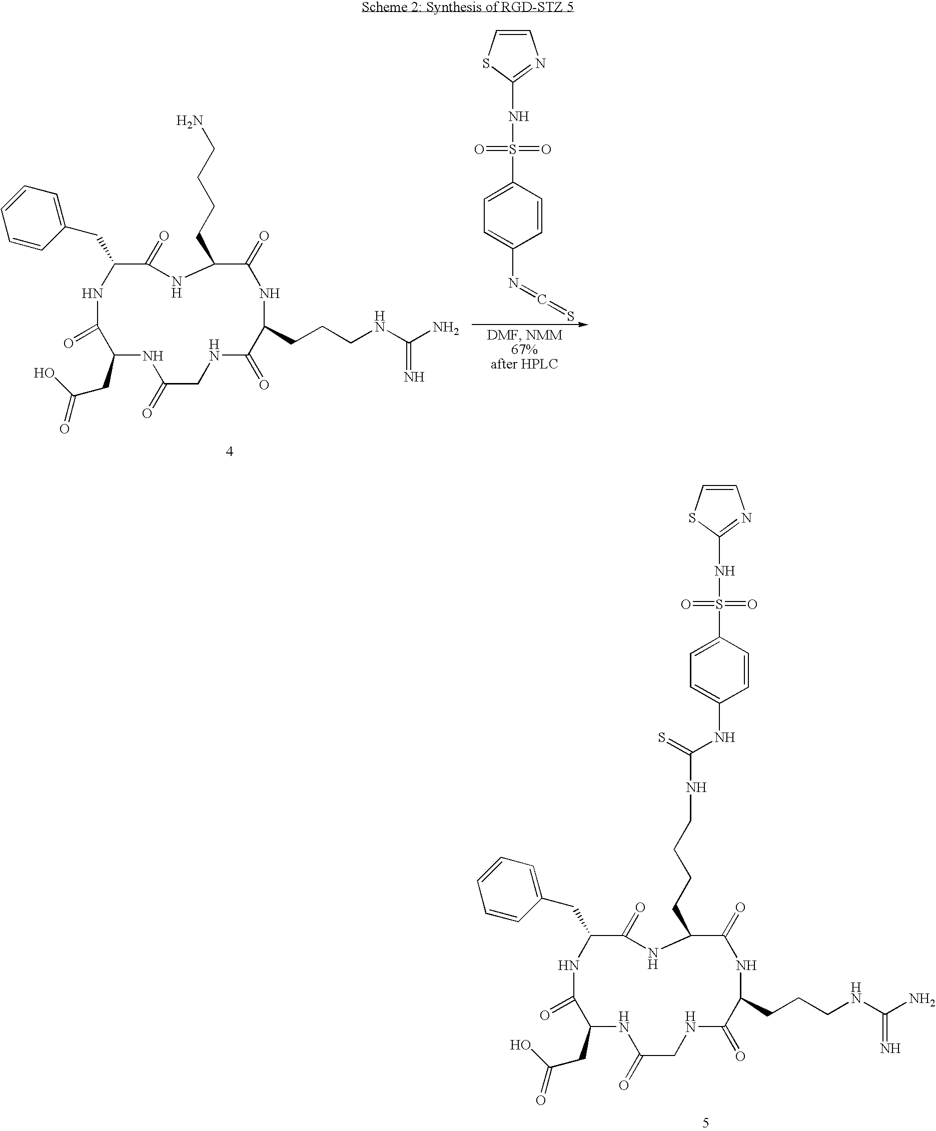

Experimental Procedures

Loading of Resin with Fmoc-Gly-OH (1):

In a Merrifield solid-phase reactor o-chlorotrityl chloride resin (3.0 g, 3.3 mmol, Novabiochem, 100-200 mesh, subst.: 1.1 mmol/g) was pre-swollen in dichloromethane (20 mL) for 30 min. Fmoc-Gly-OH (2.5 g, 8.41 mmol, 2.55 equiv.) was dissolved in a mixture of dry dichloromethane (35 mL) and dry dimethylformamide (2.5 mL), and diisopropylethylamine (1.25 mL, 8.91 mmol) was added. The solution was transferred to the reaction vessel containing the resin and the mixture was shaken for 2 h. Subsequently, the resin was washed with DMF (3×20 mL), dichloromethane (3×20 mL), methanol (3×20 mL), and diethylether (3×20 mL), and dried in vacuo to afford the loaded polymer 1 (4.17 g). The loading was determined by UV-absorption of the fluorenylmethyl-piperidine-adduct formed by treating the loaded resin (10 mg) with piperidine. Loading: c=0.845 mmol/g.

### O-tert-Butyl-L-aspartyl-D-phenylalanyl-N-tert-butoxycarboyl-L-lysyl-N-(2,2,5,7,8-pentamethyl-chroman-6-sulfonyl)-L-argininyl-glycine (2) (D(OtBu)-f-K(Boc)-R(Pmc)-G)

In an automated peptide synthesizer (Perkin Elmer ABI 433A) the target sequence was assembled according to a pre-defined coupling protocol (FastMoc 0.25) using Fmoc-Gly-OH preloaded o-chlorotrityl resin 1 (296 mg, 0.25 mmol) and the amino acid building blocks Fmoc-Asp(OtBu)-OH, Fmoc-D-Phe-OH, Fmoc-Lys(Boc)-OH, Fmoc-Arg(Pmc)-OH and Fmoc-Gly-OH. In iterative coupling cycles amino acids were sequentially attached. In every coupling step, the N-terminal Fmoc-group was removed by three 2.5 min treatments with 20% piperidine in NMP. Amino acid couplings were performed using the Fmoc-protected amino acids (1 mmol, 4 equiv.) activated by HBTU/HOBt^{[3]} (1 mmol each) and DIPEA (2 mmol) in DMF (20-30 min vortex). After every coupling step, unreacted amino groups were capped by treatment with a mixture of Ac₂O (0.5 M), DIPEA (0.125 M) and HOBt (0.015 M) in NMP (10 min vortex). Following the completion of the peptide sequence, the terminal Fmoc group was removed with 20% piperidine in NMP. The resin was thoroughly washed with NMP and dichloromethane, and transferred into a Merrifield glass reactor. The linear peptide was liberated from the solid support without affecting the acid-labile side-chain protecting groups by treating the resin with a mixture of dichloromethane, 2,2,2-trifluoroethanol (TFE), and acetic acid (15 mL, 3:1:1) for 70 min at room temperature. The resin was washed twice with the same mixture (10 mL) and subsequently with dichloromethane (2×10 mL). The combined organic phases were concentrated in vacuo and co-evaporated with toluene (3×25 mL) to give the protected, linear RGD peptide 2 as slightly yellow solid (169 mg, 0.162 mmol, 65%), which was used for the subsequent cyclization without further purification. MALDI-TOF-MS (heca, positive ion mode): calcd. for C₅₀H₇₈N₉O₁₃S: 1044.54, found: 1044.88 [M+H]⁺.

### cyclo(-R(Pmc)-G-D(OtBu)-f-K(Boc)) (3)

Under argon, a solution of the protected linear RGD peptide 2 (100 mg, 0.096 mmol) in dichloromethane (10 mL) was added slowly to a solution of 1-propanephosphonic acid cyclic anhydride (285 μL, 0.479 mmol, 5 equiv., 50% solution in ethyl acetate), triethylamine (355 μL, 2.55 mmol), and 4-di(methylamino)pyridine (2 mg) in dichloromethane (60 mL). After stirring for 18 h at room temperature, the reaction mixture was concentrated and the resulting crude product was purified by silica-column chromatography (eluent: ethyl acetate:methanol, 9:1) to afford 3 as a colorless amorphous solid (67 mg, 0.065 mmol, 68%). MALDI-TOF-MS (hcca, positive ion mode): calcd. for C₅₀H₇₅N₉O₁₂SNa: 1048.52, found: 1048.29 [M+Na]⁺; 1064.29 [M+K]⁺, clacd.: 1064.49.

### cyclo(-R-G-D-f-K) (4) (RGD)

The cyclic pentapeptide 3 (65 mg, 0.063 mmol) was dissolved in a mixture of trifluoroacetic acid (3 mL) and water (0.3 mL) and stirred for 2 h at room temperature. The reaction mixture was diluted with toluene (25 mL), concentrated in vacuo and co-evaporated with toluene (2×25 mL). The deprotected cyclopeptide was precipitated by the addition of cold diethylether (15 mL), washed three times with diethylether (15 mL), and dried under vacuum. The crude peptide was purified by preparative RP-HPLC (column: Phenomenex Jupiter Proteo 90Å, 250×10 mm) using a water:acetonitrile gradient containing 0.1% TFA to give 4 as colorless amorphous solid (29 mg, 0.048 mmol, 76%) after lyophilization. ¹H NMR (500 MHz, CD₃OD) δ 7.32-7.27 (m, 2H, f^{arom}), 7.26-7.19 (m, 3H, f^{arom}), 4.77 (t, 1H, D^{α}, J_{Dα,Dβ}=7.5 Hz), 4.50 (t, 1H, f^{α}, J_{fα,fβ}=8.5 Hz), 4.32-4.25 (m, 3H, R^{α}{4.30}, G^{αa} {4.28}), 4.01 (m, 1H, K^{α}), 3.24-3.13 (m, 3H, R^{δ}, G^{αβ}), 3.00 (d, 2H, f^{β}, J_{fβ,fα}=8.4 Hz), 2.85-2.76 (m, 3H, K^{ε}, D^{βa}), 2.61 (dd, 1H, D^{βb}, JDβa,Dβb=16.4 Hz, JDβa,Dα=5.6 Hz), 1.94-1.82 (m, 1H, R^{βa}), 1.80-1.70 (m, 1H, K^{βa}), 1.69-1.60 (m, 1H, R^{βb}), 1.60-1.38 (m, 5H, R^{γ} {1.56}, K^{δ} {1.50}, K^{βb} {1.44}), 1.01-0.92 (m, 2H, K^{δ}); ESI-HRMS: calcd. for C₂₇H₄₂N₉O₇: 604.3207, found: 604.3204 [M+H]⁺.

### cyclo(-R-G-D-f-K(STZ)) (5) (RGD-STZ)

To a solution of cyclo(-R-G-D-f-K) 4 (10 mg, 0.0166 mmol) and 4-isothiocyanato-N-thiozol2-yl-benzenesulfonamide (6 mg, 0.018 mmol, 90% purity) in DMF (5 mL) was added N-methylmorpholine (10 μL, 0.091 mmol). After stirring for 3 h at room temperature, the mixture was concentrated in vacuo and the residue was purified by preparative RP-HPLC (column: Phenomenex Jupiter Proteo 90Å, 250×10 mm) using a water:acetonitrile gradient containing 0.1% TFA to afford 5 as colorless amorphous solid (10 mg, 0.0111 mmol, 67%) after freeze-drying. ¹H NMR (600 MHz, DMSO-d₄) δ 8.42 (dd, 1H, G^{NH}), 8.11-8.02 (m, 3H, D^{NH} {8.08}, K^{NH} {8.05}, K^{ε-NH}), 8.01 (d, 1H, f^{NH}, J_{NH,fα}=6.3 Hz), 7.72-7.69 (m, 2H, STZ^{ar}), 7.63-7.60 (m, 2H, STZ^{ar}), 7.58 (d, 1H, R^{NH}, J_{NH,Rα}=7.8 Hz), 7.44 (t, 1H, R^{Gua-NH}, J_{NH,Rδ}=5.9 Hz), 7.27-7.21 (m, 3H, f^{ar}, STZ^{thiozole}), 7.19-7.13 (m, 3H, f^{ar}), 6.81 (d, 1H, STZ^{thiazole}, J=7.2 Hz), 4.65-4.60 (m, 1H, D^{α}), 4.46-4.39 (m, 1H, f^{α}), 4.16-4.11 (m, 1H, R^{α}), 4.03 (dd, 1H, G^{αa}, J_{Gαa,Gαb}=15.2 Hz, J_{Gαa,NH}=9.7 Hz), 3.96-3.90 (m, 1H, K^{α}), 3.23 (dd, 1H, G^{αb}, J_{Gαb,Gαa}=15.1 Hz, J_{Gαb,NH}=4.6 Hz), 3.11-3.04 (m, 2H, R^{δ}), 2.90 (dd, 1H, f^{βa}, J_{fαa,fαb}=13.7 Hz, J_{fαa,NH}=8.4 Hz), 2.81 (dd, 1H, f^{βb}, J_{fαb,fαa}=13.4 Hz, J_{fαb,NH}=5.9 Hz), 2.73 (dd, 1H, D^{βa}, J_{Dαa,fαb}=16.3 Hz, J_{Dαa,NH}=8.6 Hz), 2.40-2.35 (dd, 1H, D^{βb}), 1.74-1.65 (m, 1H, R^{βa}), 1.61-1.52 (m, 1H, K^{βa}), 1.52-1.29 (m, 5H, K^{βb}, R^{γ}, K^{δ),} 1.10-0.96 (m, 2H, K^{δ}); MALDI-TOF-MS (hcca, positive ion mode): calcd. for C₃₇H₄₉N₁₂O₉S₃: 901.29, found: 901.44 [M+H]⁺; 923.44 [M+Na]⁺, calcd. 923.27; 939.39 [M+K]⁺, calcd. 939.24.

Mice Immunization

Immunization of mice with dextran-bound sulfathiazole (STZ) yielded IgM and IgG antibodies. 5 BALB/c mice were immunized with STZ-Dextran with or without Freunds adjuvant. 3 mice were given 50 μg of antigen in PBS (200 μl total vol.) via a intra-peritoneal injection. 2 mice were vaccinated with 50 μg of antigen in 200 μl of formulation with complete Freund adjuvant (complete Freund adjuvant mixed 1:1 with incomplete Freund adjuvant and then 1:1 with antigen in PBS) also via a intra-peritoneal injection. Test bleeds were taken on days 5 and 10 after immunization and the final bleed was made on day 15. Approximately equal levels of IgM and IgG antibodies specific for the STZ hapten were detected by ELISA using plates coated with a STZ-BSA conjugate.

ELISA Detection of RGD-STZ Ligand Mediated Complex Formation

ELISA experiments using plates coated with purified αᵥβ₃ integrin showed excellent indirect recognition of the integrin by anti-STZ antibodies mediated by RGD-STZ. Indirect recognition of integrin by streptavidin mediated by RGD-biotin was used as positive control.

ELISA on Purified Receptor

96 well polystyrene plates (Nunc) were coated with 1 μg/ml of purified integrin αᵥβ₃ (Chemicon) in buffer: 50 mM Hepes , 0.1 M NaCl, 2 mM CaCl₂, 1 mM MnCl₂, 1 mM MgCl₂ pH 7.5. Blocking was performed with 3% BSA in the same buffer for at least 1 hr.

Ternary complex formation was obtained in concurrent or sequential mode. For sequential mode experiment the ELISA plate was incubated with RGD-STZ (root of ten dilutions starting from 10 μg/ml); 2 hr, room temperature, followed by anti-STZ rabbit serum diluted 3000 times, 1 hr RT. In the concurrent mode the incubation mixture contained both RGD-STZ and anti-STZ rabbit serum. Serum concentration was kept constant (1/3000) while RGD-STZ concentration was varied as before; incubation time 2 hr. The formed complex was detected with goat anti-Rabbit HRP conjugate. Dilutions were made in same buffer supplemented with 0.05% Tween and 0.1% BSA. BSA was not included in the washing steps.

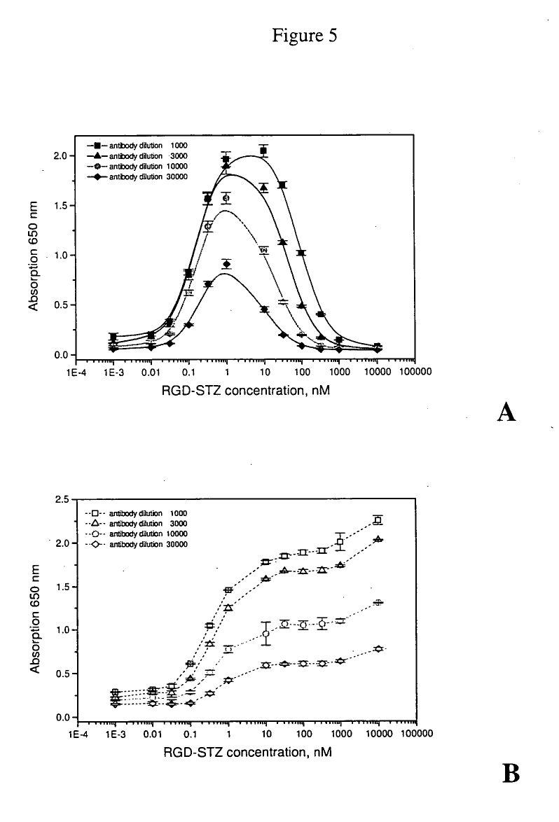

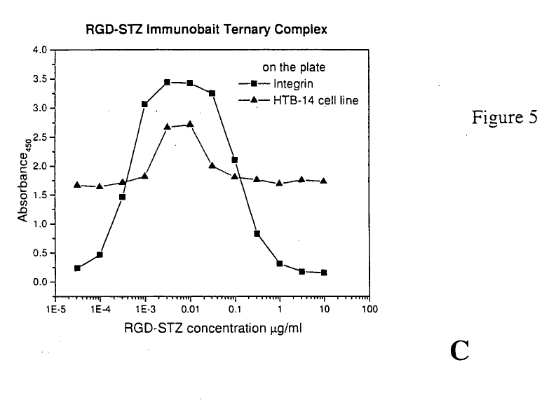

CELISA was performed in the same way on cells dried on to the culture plate. FIG. 5C demonstrates that the cell ELISA registers a similar therapeutic range of concentrations for the RGD-STZ ligands as were determined ELISA with the purified receptor.

Immunostaining

Cells could be stained through the ligand mediated association of antibody to the integrin molecule on the cell. Ligand-mediated staining of HTB-14 cells shows a distinct pattern of dots on plasma membrane as well as general fluorescent illumination of cells.

Integrin Model Discussion

ELISA Experiments using plates coated with purified αᵥβ₃ integrin show excellent indirect recognition of the integrin by anti-STZ antibodies mediated by RGD-STZ. Solid-phase assays were conducted in two different formats: concurrent and sequential incubation. In the concurrent format, RGD-STZ was premixed with rabbit serum 1E6 and incubated on the plate, which was pre-coated with integrin αᵥβ₃. In the sequential format, RGD-STZ was incubated on the integrin coated plate, washed and rabbit serum 1E6 was applied. In both formats, the signal was developed by detection of rabbit antibodies with anti-rabbit HRP antibody conjugate. Both assays have demonstrated a specific concentration-dependent RGD-STZ requirement for antibody binding to integrin-coated wells. The bell-shaped concentration dependencies (FIG. 5A) in the case of concurrent incubation clearly demonstrate the range of ternary complex stability, which broadens with higher antibody concentrations. These results show the destruction of ternary complex formation above threshold IBAIT concentrations as the formation of binary complexes (IBAIT/receptor and IBAIT/antibody) become favoured and hence do not produce a signal. This range of IBAIT concentrations in which a stable ternary complex is produced constitutes a therapeutic window, where the IBAIT can be applied for targeting immunoglobulins to malignancies.

In contrast to the concurrent format, sequential incubation resulted in a signal that steadily increased with increasing RGD-STZ (IBAIT) concentration: RGD-STZ is not present during the second incubation, and therefore, it does not tend to form binary complexes that do not produce the signal (FIG. 5B). That is, the sequential addition of antibody results in the continued formation of stable ternary complexes until receptor saturation plateaus. These results demonstrate an in vitro assay using concurrent incubation can be used to establish a therapeutic window within which an optimal range of IBAIT concentration may be determined in which a formation of a stable ternary complex (ie receptor, IBAIT, antibody) is achieved.

Immunostaining

Expression of integrin αᵥβ₃ on the surface of cultured cancer cells was confirmed using monoclonal antibody L609 and Rabbit anti-mouse Alexa fluor488. CRL-1619, HTB-14, CCL-121 appeared to be integrin positive whereas M21-L and CCL-185 (A549) were negative.

Ligand-mediated staining of HTB-14 cells was performed by incubations in sequential order. Cell were first treated with RGD-STZ then fixed with formaldehyde and stained using anti-STZ serum. The staining shows a distinct pattern of dots on the plasma membrane as well as general fluorescent illumination of cells.

Integrin αᵥβ₃ Staining

Cells were cultivated in chambers formed on microscope cover glasses with Press-to-Seal silicone isolator (Molecular Probes)—wells dimensions: 9 mm diameter 1 mm deep. Chambered cover glasses were placed in 6 well tissue culture plates.

Staining was performed with standard immunofluorescence protocol. Medium was removed by suction with a needle connected to vacuum line, cells rinsed with PBS, fixed with 10% paraformaldehyde in PBS at room temperature. Fixing was followed by 3 rinses with PBS and blocking in PBS containing 1% BSA, 2%, 0.05% Tween, 0.05% NaN₃ of heat inactivated goat serum for 40 min to 1 hr. Then, cells were incubated for 1 hr. with 10 μg/ml of monoclonal antibody against αᵥβ₃ (clone LM609—Chemicon) in blocking solution. After incubation 4 washes (5 min) with PBS were given and specimen incubated with 10 μg/ml of Goat anti-Mouse Alexa₄₈₈ in blocking solution, 4 washes with PBS (5 min) were given to remove unbound antibody. Silicone separators were finally removed and glasses mounted on microscope slides with ProLong Gold with DAPI mounting media (from Molecular Probes).

Staining of Cells via RGD-STZ and anti-STZ Complex

Cells were cultivated as described above. For staining, 6 well tissue culture plate containing chambered cover glasses was placed on ice and medium (DMEM, 10% FBS) replaced with same medium containing mixture of RGD-STZ and anti-STZ serum premixed at proportion giving highest signal in ELISA assay (˜10 μg of RGD-STZ in ml of rabbit serum). Mixture of RGD-STZ and serum was diluted in medium 30, 300 and 3000 times.

After 30 min of incubation specimens were washed with ice cold medium 3 times and incubated with Goat anti-Rabbit Alexa₄₈₈ at concentration 10 μg/ml in DMEM. After 3 rinses with ice cold DMEM cells were fixed with 10% paraformaldehyde in PBS at room temperature, washed with PBS and mounted as above.

In some experiments cells were primed with RGD-STZ (50 μg/ml in DMEM) on ice for 20 min. then fixed with paraformaldehyde and stained further like described in first protocol.

Mice Immunization with STZ-Dextran

5 BALB/c mice were immunized with STZ-Dextran conjugate with or without adjuvant. 3 mice were given 50 μg of antigen in PBS (200 μl total vol.) through intra-peritoneal injection. 2 mice were vaccinated with 50 μg of antigen in 200 μl of formulation with complete Freund adjuvant (complete Freund adjuvant mixed 1:1 with incomplete Freund adjuvant and then 1:1 with antigen in PBS) also through intra-peritoneal injection. Test bleeds were taken 4 and 8 days after immunization and final bleed on day 13.

### Example 2

### CD22 Model

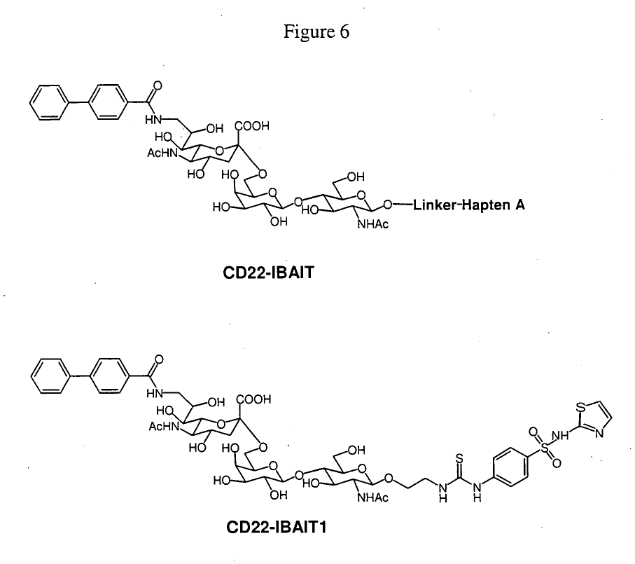

B cell lymphomas over-express cluster of antigens, including CD19, CD20, CD21, and CD22. CD22 is a sialoglycoprotein, which binds an alpha 2,6-linked sialic acid-containing glycan, as shown in FIG. 6. Analogous to the integrin model, the RGD binding motif is replaced by a trisaccharide, 8-amino-8-deoxy-8-N-(4-phenyl)phenylacetyl-N-acetyl-neuraminyl-α-(2-6)-N-acetyl-lactosylamine, having a high affinity for the CD22 cell surface antigen cluster.

### Example 4

### HN Model

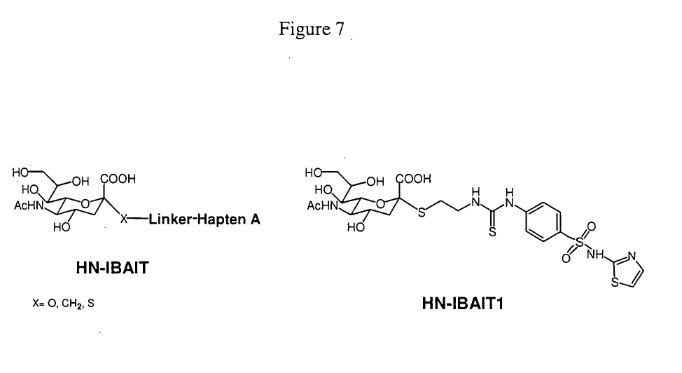

FIG. 7 shows a neuraminic acid derivative, hemagglutinin-neuraminidase (HN) known to have an affinity for viral lectins. This compound facilitates detection of viral particles by the immune system.

### Example 5

### Compound A Synthesis

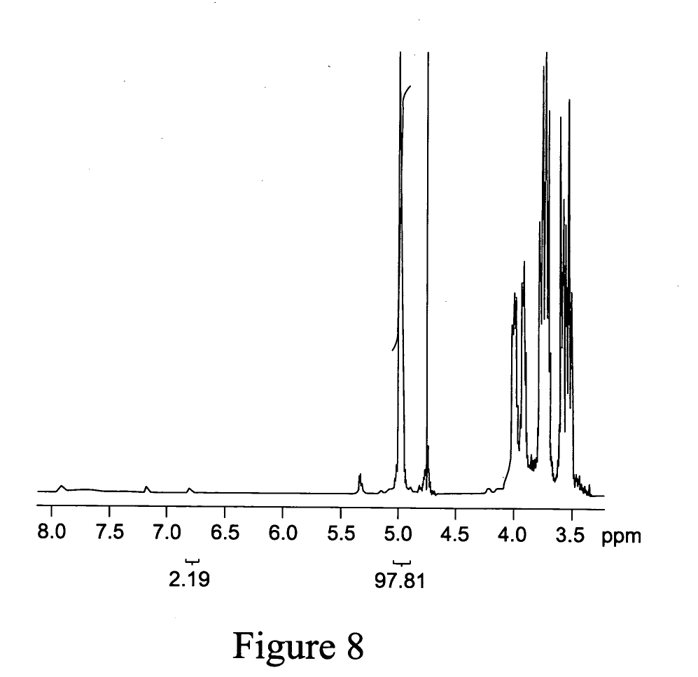

An isothiocyanate derivative of STZ (36.6 mg, 0.1 eq) was added to a solution of dextran (200 mg, 1.23 mmol per monomer, Sigma D5376, MW˜2,000,000) in DMSO (1.5 mL) and Py (5 mL) at about room temperature and stirred for about 24 hours at approximately 100° C. NCS derivative was added again and stirring continued for several hours. The mixture was dialyzed against water and centrifuged. The supernatant was freeze-dried, passed through a gel filtration column (Sephacryrl S400), and eluted with water. NMR indicated that the higher molecular weight fractions had 1% incorporation of STZ per glucose unit as shown in FIG. 8.

Discussion

The IBAIT methodologies and system described herein provide several advantages over past methodologies, namely, active and passive cancer vaccinations. Specifically, the development of synthetic drug analogues targeting cell receptors is inherently easier and cheaper than developing humanized IgG clones. Still further, the possibility of immune reaction to even humanized IgG can never be ruled out making long term treatment problematic. At the same time, active immunization strategies are hampered by intrinsic tolerance to self antigens compounded by enzymic or chemical instability of antigens.

Target antigens do not need to be unique to cancer with the IBAIT system. That is, there is no need for the cancer target to be an absolutely unique cell surface receptor, but rather provide a critical threshold response to the target cell modulated by a multimeric effect. The expression of receptor clusters on the target cells, and the mediation of this multivalency interaction by the IBAIT dose, together provides a check and balance in effecting complement cytotoxicity toward the target cells.

IBAIT system immunization allows maintenance of a strong immunological potential through vaccination, instead of the more troublesome, passive immunization alternative. That is, patient immunity is based on immunization with a synthetic ligand-dextran vaccine to develop a generic immune potential, ultimately mediated by the IBAIT to bind the target cells. Antibody titres of the IBAIT system can be maintained with the vaccine to establish a continued circulating antibody concentration during the treatment. Direct cross-reactivity of these polyclonal IgM antibodies to normal tissues is unlikely because the synthetic ligand is substantially foreign and unnatural.

Further still, the IBAIT is capable of being controlled and turned on and off. The clearance of effects of passive immunization can be a problem, as a result of the long initial infusion time, maintaining proper levels, and also in stopping problematic cross-reactive side reactions (i.e. passively infused IgG cannot be easily removed once administered). On the other hand, a small molecular weight drug of the IBAIT system can have a relatively short half life and be injected regularly over a treatment period. Injections can be stopped once treatment is complete and effective, or if there are side reactions to other tissues. Ligand mediation of the IBAIT system allows for switching the therapy on and off in a dose dependent fashion.

Further still, the IBAIT system can accommodate the screening and optimization of the target receptor. Once the patient is immunized to the antibody-binding portion of IBAIT, then the immunity allows for a customization stage. Registered IBAIT compounds, targeting a variety of different cell surface receptors, can be tested in vitro with the patient's disease, both to confirm the antibody titre and the best receptor target combination. This acknowledges the individuality of diseases and allows the optimization of the treatment for a particular disease in each patient. A differential list of candidate ligands can be generated and approved for treatments and these can be simultaneously tested on the patient's disease in vitro with the patient's own serum system (already immunized to the synthetic ligand and containing the complement effector system inherent in that patient). Once the safety of compound A is confirmed in clinical trials, pre-emptive immunization may be offered to potential patients to enable prompt commencement of treatment without a waiting induction period for an immune response against synthetic ligand 1 to develop.

The IBAIT system further enables high throughput screening potential. As in in vitro research drug development, the IBAIT system can enable high throughput screening of many heteromultivalent analogues to optimize linking arm type length, etc.

The above-described embodiments of the present invention are intended to be examples only. Alterations, modifications and variations may be effected to the particular embodiments by those of skill in the art without departing from the scope of the invention, which is defined solely by the claims appended hereto.

### REFERENCES

[1] L. A. Carpino, G. Y. Han, J. Am. Chem. Soc. 1970, 92, 5748.
[2] X. Dai, Z. Su, J. O. Liu, Tetrahdron Lett. 2000, 41, 6225-6298.
[3] L. A. Carpino, H. Imazumi, A. El-Faham, F. J. Ferrer, C. Zhang, Y. Lee, B. M. Foxmann, P. Henklein, C. Hanay, C. Mügge, H. Wenschuh, J. Klose, M. Beyermann, M. Bienert, Angew. Chem. Int. Ed. 2002, 41, 441.

1. A method of targeted immunotherapy comprising administering an effective amount of a compound B having a receptor binding factor (RBF), a synthetic hapten ligand and a linker molecule connecting the RBF and synthetic hapten ligand wherein administration of compound B initiates immune recognition of compound B by pre-existing heterovalent antibodies and wherein the RBF binds a surface receptor of a target and the heterovalent antibodies bind the synthetic hapten ligand.

2. A method as in claim 1 wherein the target are target cells and compound B promotes antibody-mediated cytotoxicity of transformed target cells.

3. A method as in claim 2 wherein compound B is administered at a threshold level determined to allow complex formation and activation of antibody-mediated cytotoxicity.

4. A method as in claim 2 wherein compound B is administered at a dose to promote a multipoint interaction between said antibodies and the target cells.

5. A method as in claim 1 wherein the pre-existing heterovalent antibodies are raised in a patient prior to commencing a treatment by administering a compound A, compound A having a carrier, a synthetic hapten ligand and a linker molecule connecting the carrier and synthetic hapten ligand.

6. A method as in claim 1 wherein the RBF is Arginine-Glycine-Aspartic Acid (RGD) or functional derivative or synthetic mimetic thereof.

7. A method as in claim 6 wherein the RGD is a cyclo-peptide.

8. A method as in claim 1 wherein the synthetic hapten ligand is a sulfonamide.

9. A method as in claim 8 wherein the synthetic ligand is sulfathiazole (STZ).

10. A method as in claim 1 wherein the linker molecule is a heteroatom substituted or un-substituted C2-C20 aliphatic chain.

11. A method as in claim 1 wherein the linker molecule is a substituted or un-substituted aromatic.

12. A method as in claim 1 wherein the linker is a polymer.

13. A method as in claim 1 wherein the carrier is a non-protein carrier selected to promote an IgM antibody response.

14. A method as in claim 1 wherein the carrier is a carbohydrate selected to promote an IgM response.

15. A method as in claim 14 wherein the carbohydrate is dextran or beta-glucan.

16. A method as in claim 5 wherein the synthetic hapten ligand is a sulfonamide.

17. A method as in claim 5 wherein the synthetic ligand is any one of nitrophenol, α-(1-3)galactosyl-lactose or ABO blood group antigens.

18. A method as in claim 16 wherein the synthetic ligand is sulfathiazole (STZ).

19. A method as in claim 5 wherein the carrier is a protein carrier and promotes raising an IgG response.

20. A method as in claim 1 wherein the pre-existing heterovalent antibodies are human anti-blood group A, B or O antibodies or anti-α-gal antibodies including xenotransplantation or Galili antigen.

21. A method as in claim 5 wherein compound A is administered at a level to maintain a minimum antibody concentration during treatment.

22. A method as in claim 1 wherein the RBF binds Integrin αvβ3 cell surface receptor.

23. A method as in claim 1 wherein the RBF binds a sialoglycoprotein associated with a B cell lymphoma.

24. A method as in claim 1 wherein the RBF is a 2,6-linked sialic acid-containing oligosaccharide.

25. A method as in claim 1 wherein the RBF is a trisaccharide.

26. A method as in claim 1 wherein the RBF is a neuraminic acid derivative.

27. A method as in claim 1 wherein the RBF binds hemagglutinin-neuraminidase (HN).

28. A method as in claim 1 wherein the RBF binds viral lectins.

29. A compound for use in immunotherapy comprising a receptor binding factor (RBF), a synthetic hapten ligand and a linker molecule connecting the RBF and synthetic hapten ligand wherein administration of the compound to a system having pre-existing heterovalent antibodies initiates immune recognition of the compound by the pre-existing heterovalent antibodies and wherein the RBF binds a surface receptor of a target and the heterovalent antibodies bind the synthetic hapten ligand.

30. A compound as in claim 29 wherein the target is a target cell and the compound promotes complement-mediated cytotoxicity of transformed cells.

31. A compound as in claim 29 wherein the RBF is Arginine-Glycine-Aspartic Acid (RGD) or a functional derivative thereof.

32. A compound as in claim 29 wherein the RGD is a cyclopeptide.

33. A compound as in claim 29 wherein the synthetic hapten ligand is a sulfonamide or a polyacrylamide.

34. A compound as in claim 29 wherein the synthetic ligand is sulfathiazole (STZ).

35. A compound as in claim 29 wherein the linker molecule a heteroatom substituted or un-substituted C2-C20 aliphatic chain.

36. A compound as in claim 29 wherein the linker molecule is a substituted or un-substituted aromatic.

37. A compound as in claim 29 wherein the linker is a polymer.

38. A compound for raising heterovalent antibodies comprising a carrier, a synthetic hapten ligand and a linker molecule connecting the carrier and synthetic hapten ligand wherein the carrier is a non-protein carrier that promotes raising an IgM antibody response.

39. A compound as in claim 38 wherein the carrier is a carbohydrate capable of raising an IgM response.

40. A compound as in claim 38 wherein the carbohydrate is dextran or beta-glucan.

41. A compound as in claim 38 wherein the synthetic ligand is a sulfonamide.

42. A compound as in claim 38 wherein the synthetic ligand is sulfathiazole (STZ).

43. A compound as in claim 38 wherein the carrier is a protein carrier that promotes raising an IgG response.

44. An assay method to determine an optimum concentration range of a compound as defined in claim 1, the optimum concentration range of the compound defining a therapeutic window for the use of the compound in immunotherapy, comprising the steps of:
a. concurrently incubating i) the compound comprising a receptor binding factor (RBF), a synthetic hapten ligand and a linker molecule connecting the RBF and synthetic hapten ligand together with ii) heterovalent antibodies and iii) an anchored target; and,
b. measuring the concentration of a formed ternary non-covalent complex, the formed ternary complex including the compound, antibody and target;
c. repeating step b) at varying compound concentration levels to determine an optimum concentration range in which the formed ternary complex is formed.

45. An assay method as in claim 44 wherein the anchored target is a target cell.

46. An assay method as in claim 44 wherein the anchored target is a purified receptor on the surface of the target cell which is able to bind the RBF of the compound.
